(19) United States
(12) Patent Application Publication
(10) Pub. No.: US 2026/0085024 A1
(43) Pub. Date: Mar. 26, 2026

(54) METHODS FOR DEHYDROGENATING HYDROCARBONS UTILIZING COUNTERCURRENT FLOW REGENERATORS

(71) Applicant: Dow Global Technologies LLC, Midland, MI (US)

(72) Inventors: Matthew T. Pretz, Lake Jackson, TX (US); Charmaine Bennett, Houston, TX (US); Kevin Blann, Missouri City, TX (US); Jordan Keith, Pearland, TX (US); Davy Nieskens, Terneuzen (NL); Anthony Plauck, Sugarland, TX (US)

(73) Assignee: Dow Global Technologies LLC, Midland, MI (US)

(21) Appl. No.: 19/111,054

(22) PCT Filed: Sep. 12, 2023
(86) PCT No.: PCT/US2023/073960

§ 371 Date: Mar. 12, 2025

Related U.S. Application Data
(60) Provisional application No. 63/406,444, filed on Sep. 14, 2022.

(51) Int. Cl. C07C 5/48

(52) U.S. Cl. CPC C07C 5/48

(57) ABSTRACT

According to one or more embodiments described herein, hydrocarbons may be dehydrogenated by a method including contacting a feed stream including one or more hydrocarbons with a particulate solid, which may include an oxygen-carrier material, to form hydrogen and one or more products. At least a portion of the hydrogen may be reacted with oxygen from the oxygen-carrier material. The particulate solid from the dehydrogenation reactor maybe passed to a regeneration unit, which may include a first gas inlet and a second gas inlet that may be beneath the first gas inlet. An oxygen-containing gas may enter into the regeneration unit through the first gas inlet and a fuel may enter through the second gas inlet. In an area of the regeneration unit above the second gas inlet and beneath the first gas inlet, at least a portion of the fuel may be reacted with oxygen from the oxygen-carrier material.

### CROSS REFERENCE TO RELATED APPLICATION

This application claims priority to U.S. Provisional Application Ser. No. 63/406,444, filed Sep. 14, 2022, which is hereby incorporated by reference in its entirety.

### TECHNICAL FIELD

Embodiments described herein generally relate to chemical processing and, more specifically, to processes and systems utilized for dehydrogenation of chemical species.

### BACKGROUND

Olefinic compounds may be utilized as base materials to produce many types of goods and materials. For example, ethylene may be utilized to manufacture polyethylene, ethylene chloride, or ethylene oxides. Such products may be utilized in product packaging, construction, textiles, etc. Thus, there is an industry demand for light olefins, such as ethylene, propylene, butene, and styrene.

### SUMMARY

One method for producing olefinic compounds is by dehydrogenating alkanes and/or alkyl aromatics. In some embodiments, the dehydrogenation reaction may be promoted by reducing or removing hydrogen formed during dehydrogenation by reacting the hydrogen with oxygen to form water, which pushes the equilibrium towards the light olefin products. In such embodiments, an oxygen-carrier material may be utilized to provide the oxygen that reacts with the hydrogen. The oxygen-carrier material may be included in a particulate solid. Such oxygen-carrier materials may cycle through a reactor and a regeneration unit, where the content of oxygen in the oxygen carrier material may be increased in the regeneration unit. In some embodiments, a fuel is additionally utilized in the regeneration unit to heat at least the oxygen carrier material.

As is described herein, it has been discovered that it may be beneficial to utilize a particular countercurrent flow pattern with respect to the particulate solid and the gases in the regeneration unit. Embodiments described herein include flow patterns where the particulate solid moves generally downwards though the regeneration unit and the gases, including the fuel and an oxygen-containing gas move in a generally upwards direction. Additionally, the oxygen-containing gas and the fuel enter the regeneration unit through first and second gas inlets, respectively, where the second gas inlet (for the fuel) is beneath the first gas inlet (for the oxygen-containing gas). In such embodiments, the particulate solid may contact the oxygen-containing gas (and sometimes fuel) prior to being later contacted with fuel (and not oxygen-containing gas). In the regeneration unit, oxygen from the oxygen-carrier material may be utilized to react with the fuel, reducing the oxygen content of the oxygen-carrier material. It may be desirable to have the oxygen content of the oxygen-carrier material be at least partially reduced when the oxygen-carrier material is passed to the dehydrogenation reactor to maintain the desired activity and selectivity of the oxygen-carrier material. The embodiments described herein may accommodate such a desire by having the oxygen-carrier material contact fuel (without additional oxygen-containing gas) after the oxygen-carrier material is contacted by the oxygen-containing gas.

According to one or more embodiments described herein, hydrocarbons may be dehydrogenated by a process comprising contacting a feed stream comprising one or more hydrocarbons with a particulate solid in a dehydrogenation reactor. The particulate solid may comprise an oxygen-carrier material. In the dehydrogenation reactor one or more hydrocarbons may be dehydrogenated to form hydrogen and one or more products. At least a portion of the hydrogen may be reacted with oxygen from the oxygen-carrier material to form water and reduce the oxygen content in the oxygen-carrier material. The process may further comprise passing the particulate solid from the dehydrogenation reactor to a regeneration unit. The particulate solid may move in a generally downward direction through the regeneration unit and gases may move in a generally upwards direction through the regeneration unit, such that the particulate solid and the gases move in a countercurrent flow pattern through the regeneration unit. The regeneration unit may comprise a first gas inlet and a second gas inlet. The second gas inlet may be beneath the first gas inlet. An oxygen-containing gas may enter into the regeneration unit through the first gas inlet. A fuel may enter into the regeneration unit through the second gas inlet. In an area of the regeneration unit above the second gas inlet and beneath the first gas inlet at least a portion of the fuel may be reacted with oxygen from the oxygen-carrier material of the particulate solid. The process may further comprise passing at least a portion of the particulate solid from the regeneration unit to the dehydrogenation unit.

It is to be understood that both the preceding general description and the following detailed description describe various embodiments and are intended to provide an overview or framework for understanding the nature and character of the claimed subject matter. Additional features and advantages of the embodiments will be set forth in the detailed description and, in part, will be readily apparent to persons of ordinary skill in the art from that description, which includes the accompanying drawings and claims, or recognized by practicing the described embodiments. The drawings are included to provide a further understanding of the embodiments and, together with the detailed description, serves to explain the principles and operations of the claimed subject matter. However, the embodiments depicted in the drawings are illustrative and exemplary in nature, and not intended to limit the claimed subject matter.

### BRIEF DESCRIPTION OF THE DRAWINGS

Reference will now be made in greater detail to various embodiments, some of which are illustrated in the accompanying drawings, wherein:

FIG. 1 schematically depicts a reactor system, according to one or more embodiments of the present disclosure;

FIG. 2 schematically depicts another reactor system according to additional embodiments of the present disclosure.

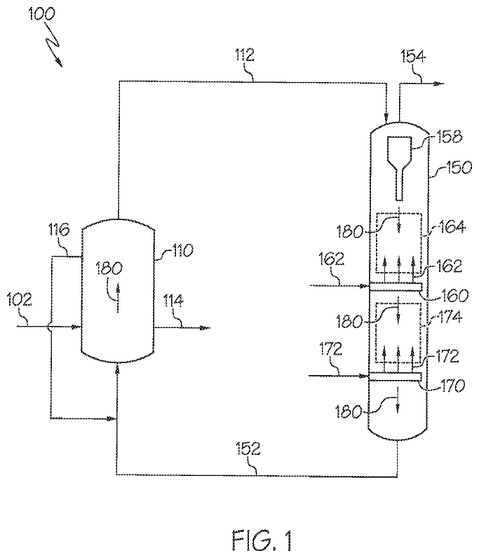

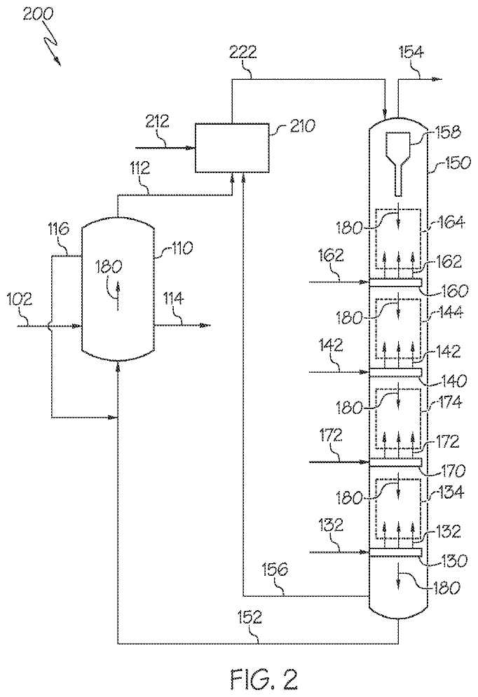

When describing the simplified schematic illustration of FIG. 1 and FIG. 2, the numerous valves, temperature sensors, electronic controllers, and the like, which may be used and are well known to a person of ordinary skill in the art, are not included. Further, accompanying components that are often included in such reactor systems, such as air supplies, heat exchangers, surge tanks, and the like are also not included. However, it should be understood that these components are within the scope of the present disclosure.

### DETAILED DESCRIPTION

Specific embodiments of the present application will now be described. The disclosure may, however, be embodied in different forms and should not be construed as limited to the embodiments set forth in this disclosure. Rather, these embodiments are provided so that this disclosure will be thorough and complete, and will fully convey the scope of the subject matter to those skilled in the art.

Embodiments of the methods presently disclosed will now be described herein in detail in the context of the reactor system of FIG. 1 and FIG. 2 operating as circulating fluidized bed processes to dehydrogenate hydrocarbons. However, it should be understood that the principles disclosed and taught herein may be applicable to other systems which utilize different system components oriented in different ways. For example, the concepts described herein may be equally applied to other systems with alternate reactor units and regeneration units, such as those that operate under non-fluidized conditions or include downers rather than risers. It should be further understood that not all portions of the reactor systems of FIG. 1 and FIG. 2 should be construed as essential to the claimed subject matter. Moreover, while the recited method steps in the appended claims are described herein in the context of the reactor systems of FIG. 1 and FIG. 2, such recited method steps should be understood as adaptable to other systems, as would be understood by those skilled in the art.

Now referring to FIG. 1, an example reactor system 100 that may be suitable for use with the methods described herein is schematically depicted. The reactor system 100 may include a dehydrogenation reactor 110 and a regeneration unit 150. A feed stream 102 may be passed into the dehydrogenation reactor 110. A particulate solid 180 may be passed into the dehydrogenation reactor 110 via stream 152. The particulate solid 180 may be contacted with the feed stream 102 in the dehydrogenation reactor 110. The particulate solid 180 may comprise an oxygen-carrier material. The feed stream 102 may comprise one or more hydrocarbons which may be dehydrogenated in the dehydrogenation reactor 110 to form one or more products and hydrogen. Oxygen from the oxygen-carrier material may react with the hydrogen to form water. The one or more products may exit the dehydrogenation reactor 110 via a product stream 114.

The particulate solid 180 may exit the dehydrogenation reactor 110 and may be passed to the regeneration unit 150 via stream 112. The regeneration unit 150 may comprise a gas/solids separator 158, a first gas inlet 160 and a second gas inlet 170. The first gas inlet 160 may be above the second gas inlet 170. An oxygen-containing gas 162 may enter into the regeneration unit 150 through the first gas inlet 160 and a fuel 172 may enter into the regeneration unit 150 through the second gas inlet 170. The particulate solid 180 may travel in a generally downwards direction through the regeneration unit 150 first through the separator 158, then past the first gas inlet 160, and then past the second gas inlet 170. The gases within the regeneration such as the oxygen-containing gas 162 and the fuel 172 may travel in a generally upwards direction through the regeneration unit 150, such that the particulate solid 180 and the gases move in a countercurrent flow pattern through the regeneration unit 150. The particulate solid 180 may then exit from the regeneration unit 150 and be passed back to the dehydrogenation reactor 110 via stream 152.

In some embodiments, reactor system 100 may be operable to perform a circulating fluidized bed (CFB) dehydrogenation process. The CFB dehydrogenation process may include a dehydrogenation reactor 110 and a regeneration unit 150, both fluid bed based.

As discussed previously, in one or more embodiments, the feed stream 102 may be passed into the dehydrogenation reactor 110. In one or more embodiments, the feed stream 102 may comprise one or more hydrocarbons. In one or more embodiments, the one or more hydrocarbons may comprise an alkyl moiety. As used in the present disclosure a hydrocarbon comprises an “alkyl moiety” if the molecule has at least one carbon-carbon single bond capable of being dehydrogenated to form a carbon-carbon double bond. In one or more embodiments, the one or more hydrocarbons may comprise one or more of ethane, propane, butane, or ethylbenzene. According to one or more embodiments, the one or more hydrocarbons may comprise at least 50 wt. %, at least 60 wt. %, at least 70 wt. %, at least 80 wt. %, at least 90 wt. %, at least 95 wt. % or even at least 99 wt. % of ethane. In additional embodiments, the one or more hydrocarbons may comprise at least 50 wt. %, at least 60 wt. %, at least 70 wt. %, at least 80 wt. %, at least 90 wt. %, at least 95 wt. % or even at least 99 wt. % of propane. In additional embodiments, the one or more hydrocarbons may comprise at least 50 wt. %, at least 60 wt. %, at least 70 wt. %, at least 80 wt. %, at least 90 wt. %, at least 95 wt. % or even at least 99 wt. % of butane. In additional embodiments, the one or more hydrocarbons may comprise at least 50 wt. %, at least 60 wt. %, at least 70 wt. %, at least 80 wt. %, at least 90 wt. %, at least 95 wt. % or even at least 99 wt. % of ethylbenzene. In additional embodiments, the one or more hydrocarbons may comprise at least 50 wt. %, at least 60 wt. %, at least 70 wt. %, at least 80 wt. %, at least 90 wt. %, at least 95 wt. % or even at least 99 wt. % of the sum of ethane, propane, butane and ethylbenzene.

According to additional embodiments, the dehydrogenation reactor 110 may operate with a “back-mixed” fashion where the feed stream 102 enters the reactor 110, as to closely approximate isothermal conditions. As such, the fluid velocity at this region may be low enough and the particulate solid 180 flux may be great enough such that a dense bed may form at or around where the feed stream 102 is injected. In some embodiments, the superficial velocity of the reactor 110 may be from 3-80 ft/s, such as from 3-40 ft/s, or 10-30 ft/s. The particulate solid 180 flux in the reactor 110 may be from 1-300 lb/ft²-s, such as from 40-200 lb/ft²-s, or from 60-160 lb/ft²-s. The reactor 110 may include multiple diameters, and may include one or more frustums to increase or decrease particulate solid 180 and/or gaseous reactant velocity. The reactor 110 may operate with a gas residence time of from 0.1-10 seconds, such as from 0.5-6 seconds.

A particulate solid 180 may be utilized in the general operation of the reactor system 100. As used herein, the term “particulate solid” may refer to one or more solid particles suitable for fluidization. In one or more embodiments, the particulate solid 180 may comprise an oxygen-carrier material and a dehydrogenation catalyst material. In some embodiments, the particulate solid 180 may consist essentially of the oxygen-carrier material. As described herein, “consists essentially of” refers to materials with less than 1 wt. % of the non-recited materials (i.e., consisting essentially of A means A is at least 99 wt. % of the composition). In some embodiments, the particulate solid 180 may not comprise a dehydrogenation catalyst material. In some embodiments, the oxygen-carrier material and the dehydrogenation catalyst material may be separate particles of the particulate solid 180. In some embodiments, the oxygen-carrier material and the dehydrogenation catalyst may be contained in the same particles of the particulate solid 180.

In embodiments where the particulate solid 180 comprises a dehydrogenation catalyst, the dehydrogenation of the one or more hydrocarbons may be at least partially by catalytic dehydrogenation. Catalytic dehydrogenation is the dehydrogenation of a hydrocarbon that is promoted by the use of a dehydrogenation catalyst. In embodiments, where the particulate solid 180 does not comprise a dehydrogenation catalyst the dehydrogenation of the one or more hydrocarbons may be by non-catalytic thermal dehydrogenation. Non-catalytic thermal dehydrogenation refers to the dehydrogenation of a hydrocarbon that occurs without the use of a dehydrogenation catalyst and instead may occur because of high temperature, pressure or combinations thereof.

In some embodiments, the particulate solid 180 may comprise a “dual-purpose material” that may act as both a dehydrogenation catalyst as well as an oxygen-carrier material. It should be understood that, in at least the embodiments described herein where an oxygen-carrier material and a dehydrogenation catalyst are utilized in the same reaction vessel (such as those of FIG. 1), such a dual-purpose material may be utilized either in replacement or in combination with the oxygen-carrier material of the particulate solid 180 or the dehydrogenation catalyst of the particulate solid 180.

Unless specified herein, an “oxygen-carrier material” may generally refer to an oxygen-rich oxygen-carrier material or an oxygen-deficient oxygen-carrier material. Unless specified herein, the “dual-purpose material” may generally refer to an oxygen-rich dual-purpose material or an oxygen-deficient dual-purpose material. For example, an oxygen-deficient state may be present after some oxygen is utilized for combustion and may be oxygen-rich prior to the combustion, following regeneration of the oxygen-deficient state material. The reactions may take place in one or more fluidized bed reactors, such as circulating fluidized bed reactors. The reactors may be, for example, risers or downers.

As described herein, in one or more embodiments, the dehydrogenation catalyst and the oxygen-carrier material may be separate particles of the particulate solid 180. One contemplated advantage of such a system is that by adding, removing, or substituting one or both of the dehydrogenation catalyst and oxygen-carrier material, the functionality of the system can be altered, even when the system is on-line. For example, the reaction heat load could be adjusted by adding or removing one or both of the dehydrogenation catalyst and the oxygen-carrier material. This may be advantageous, in some embodiments, as compared with a dual purpose material, since the dual purpose particle's heat balance must be determined prior to reaction and cannot be easily adjusted by varying the amount of dehydrogenation catalyst versus oxygen-carrier material. Control of the ratio of dehydrogenation catalyst versus oxygen-carrier material may further be advantageous since reaction selectivity may be better tuned. For example, the amount of hydrogen in the system may be used to control the degree of combustion, or component balances may be used to optimize downstream separation processes.

In some embodiments, the dehydrogenation reactor 110 may include from 1 wt. % to 100 wt. %, such as from 5 wt. % to 95 wt. %, or from 75 wt. % to 25 wt. %, oxygen-carrier material based on the total weight of the particulate solid 180 in the dehydrogenation reactor 110. In other embodiments, the dehydrogenation reactor 110 may include from 50 wt. % to 75 wt. % oxygen-carrier material based on the total weight of the particulate solid 180 in the dehydrogenation reactor 110. In some embodiments, relatively large amounts of oxygen-carrier material may be present (e.g., at least 80 wt. %, at least 85 wt. %, or even at least 90 wt. %), particularly if there is relatively slow release of oxygen by the oxygen-carrier materials as compared to the rate of dehydrogenation. In some embodiments, the dehydrogenation reactor 110 may include from 0 wt. % to 99 wt. %, such as from 5 wt. % to 95 wt. %, or from 25 wt. % to 75 wt. %, dehydrogenation catalyst based on the total weight of the particulate solid 180 in the dehydrogenation reactor 110. In other embodiments, the dehydrogenation reactor 110 may include from 25 wt. % to 50 wt. % dehydrogenation catalyst based on the total weight of the particulate solid 180 in the dehydrogenation reactor 110. In some embodiments, the dehydrogenation reactor 110 may include up to 95 wt. %, 99 wt. %, or even 100 wt. % of dual-purpose material of the total weight of the particulate solid 180 in the dehydrogenation reactor 110. For example, all or a far majority of the particulate solid 180 may be dual-purpose material when utilized. In embodiments, the particulate solid 180 material may encompass all solids in the system aside from coke.

As stated previously, within the dehydrogenation reactor 110, hydrogen may be contacted with the oxygen-rich oxygen-carrier material of the particulate solid 180. The oxygen-carrier material may include one or more metal oxides. According to one or more embodiments, the one or more metal oxides may be a redox-active metal oxide or a mixture of redox-active metal oxides. The redox-active metal oxide includes binary, ternary, or other mixed metal oxides capable of undergoing reduction in the presence of a reducing agent (for example, hydrogen) and oxidation in the presence of oxidizing agent (for example, oxygen or air). In some embodiments, the redox-active metal oxide may be a metal MOₓ, where M may be one or more metals of IUPAC group 6, 7, 8, 9, 10, 11, or 12 and “x” is the number of associated oxygen atoms in the structure. For example the redox-active metal oxide may be Mn₂O₃, Fe₂O₃, Co₃O₄, CuO, (LaSr)CoO₃, (LaSr)MnO₃, Mg₆MnO₈, MgMnO₃, MnO₂, Fe₃O₄, Mn₃O₄, Cu₂O, NiO, Ni₂O₃, CrO, Cr₂O₃, CrO₂, ZnO, or any combination of other IUPAC group 6-12 metal oxide. In some embodiments, the redox-active metal oxide may be cerium oxide. For example, the redox-active metal oxide may be CeO2, Ce₂O₃, or any other mixed metal oxide containing cerium. In further embodiments, the oxygen carrier material may include lanthanum oxide, La₂O₃, in combination with other reducible metal oxides. In some embodiments, the redox-active metal oxide may be chosen from Mn₂O₃, Fe₂O₃, Co₃O₄, CuO, (LaSr)CoO₃, (LaSr)MnO₃, Mg₆MnO₈, MgMnO₃, MnO₂, Fe₃O₄, Mn₃O₄, and Cu₂O. In some embodiments, the oxygen-carrier material may be a solid. In specific embodiments, the oxygen-carrier material may be a crushed solid or powder. In other embodiments, the oxygen-carrier material may be formulate using a redox-active metal oxide and a binder and/or support material to produce a fluidizable material with the require physical properties, for example, particle size distribution, density, and attrition resistance. The binder and/or support material may include alumina, silica, titania, magnesia, zirconia, or combinations thereof.

In one or more embodiments, the oxygen-carrier material may include a hydrogen-selective oxygen-carrier material that may include a promoter or a combination of various promoters. The addition of a promoter(s) may lead to the formation of a core-shell morphology. The promoter(s) may include alkali or alkaline-earth metal oxides from IUPAC group 1 and 2 and/or compounds comprising alkali-transition metal oxides or alkaline-earth transition metal oxides. In some embodiments, alkali elements may include one or more of sodium, lithium, potassium, and cesium. In some embodiments, alkaline-earth elements may include one or more of calcium, magnesium, strontium, and barium. In some embodiments, transition metals may include one or more of tungsten and molybdenum. For example, the one or more alkali or alkaline-earth transition metal oxides may be Na₂WO₄, K₂MoO₄, Na₂MoO₄, K₂WO₄, Li₂WO₄, CsWO₄, Li₂MoO₄, CaWO₄, CaMoO₄, MgWO₄, MgMoO₄, SrWO₄, SrMoO₄, BaWO4 and BaMoO₄. In some embodiments, the promoter may include one or more of alkali or alkaline-earth metal salts selected from Group 1 and 2 metal cations and a counterion. In some embodiments, alkali elements may include one or more of sodium, lithium, potassium, and cesium. In some embodiments, alkaline-earth elements may include one or more of calcium, magnesium, strontium, and barium. In some embodiments, the counterion may include carbonates, sulphates, sulphites, sulfides, phosphates, phosphites and borates. For example, the alkali or alkaline-earth metal salts may be Na₂CO₃, Na₂SO₄, Na₃PO₄, Li₂CO₃, Li₂SO₄, Li₃PO₄, K₂CO₃, K₂SO₄, K₃PO₄, Cs₂CO₃, Cs₂SO₄, Cs₃PO₄, CaCO₃, CaSO₄, Ca₃(PO₄)₂, SrCO₃, SrSO₄, Sr₃(PO₄)₂, MgCO₃, MgSO₄, Mg₃(PO₄)₂, BaCO₃, BaSO₄, Ba₃(PO₄)₂, Na₂HPO₄, KHSO₄, Na₂SO₃, K₂B₄O₇, Na₃BO₃, or combinations thereof.

For example, oxygen-carrier materials such as those disclosed in U.S. App. No. 62/725,504, entitled “METHODS OF PRODUCING HYDROGEN-SELECTIVE OXYGEN CARRIER MATERIALS,” filed on, Aug. 31, 2018, and U.S. App. No. 62/725,508, entitled “HYDROGEN-SELECTIVE OXYGEN CARRIER MATERIALS AND METHODS OF USE,” filed on, Aug. 31, 2018, are contemplated as suitable for the presently disclosed processes, and the teachings of these references are incorporated by reference herein. In one or more additional embodiments, the oxygen-carrier material may include those of U.S. Pat. Nos. 5,430,209, 7,122,495, and/or WO 2018/232133, each of which are incorporated by reference in their entireties.

The oxygen-rich oxygen-carrier material may be reducible by releasing oxygen that may be selective for combusting hydrogen. For example, the oxygen-carrier material may be selective for the combustion of hydrogen over hydrocarbons. In some embodiments, the oxygen-rich oxygen-carrier material comprises from about 1 wt. % to about 20 wt. % releasable oxygen based on total weight of the oxygen-rich oxygen-carrier material. In other embodiments, the oxygen-rich oxygen-carrier material comprises from about 1 wt. % to about 10 wt. %, from about 1 wt. % to about 5 wt. %, from about 5 wt. % to about 20 wt. %, or from about 5 wt. % to about 10 wt. % releasable oxygen. As described herein, “releasable oxygen” may refer to the oxygen that can be released through redox by the oxygen-carrier material. Other oxygen may be present in the oxygen-carrier material that is not releasable through redox. It should be understood that in some embodiments, the oxygen may be released from a surface of the oxygen-carrier material simultaneously with the combustion of hydrogen at the surface of the oxygen-carrier material.

As stated previously, the releasable oxygen of the oxygen-rich oxygen-carrier materials may be selective for combusting hydrogen over hydrocarbons. In some embodiments, at least about 60% of the releasable oxygen of the oxygen-carrier material is selective for hydrogen combustion. In other embodiments, at least about 55% of the releasable oxygen of the oxygen-carrier material is selective for hydrogen combustion.

In embodiments, when the hydrogen is contacted by the oxygen-rich oxygen-carrier material, some of the releasable oxygen is removed from the oxygen-rich oxygen-carrier material. In some embodiments, contacting the hydrogen with the oxygen-rich oxygen-carrier material removes from about 1 wt. % to 50 wt. % of the releasable oxygen from the oxygen-rich oxygen-carrier material. In other embodiments, contacting the hydrogen with the oxygen-rich oxygen-carrier material removes from about 10 wt. % to about 50 wt. %, from about 10 wt. % to about 25 wt. %, or from about 25 wt. % to about 50 wt. % of the releasable oxygen from the oxygen-rich oxygen-carrier material.

In further embodiments, when the hydrogen is contacted by the oxygen-rich oxygen-carrier material, the oxygen-rich oxygen-carrier material combusts greater than about 50% of the hydrogen. In other embodiments, when the hydrogen is contacted by the oxygen-rich oxygen-carrier material, the oxygen-rich oxygen-carrier material combusts about 50% to about 90%, or about 75% to about 90% of the hydrogen that is produced.

The contacting of the oxygen-rich oxygen-carrier material with the hydrogen may combust the hydrogen and form an oxygen-diminished oxygen-carrier material. To form the oxygen-diminished oxygen-carrier material, at least a portion of the oxygen-rich oxygen-carrier material may be reduced to a lower oxidation state. Once the oxygen-carrier material has been reduced to form the oxygen-diminished oxygen-carrier material, the oxygen-diminished oxygen-carrier material may be discharged from the dehydrogenation reactor 110 at a lower oxidation state.

As described herein, in one or more embodiments, the particulate solid 180 may comprise a dehydrogenation catalyst. In one or more embodiments, the dehydrogenation catalyst may include gallium, chromium, and/or platinum. As described herein, a gallium and/or platinum dehydrogenation catalyst comprises gallium, platinum, or both. The dehydrogenation catalyst may be carried by an alumina or alumina silica support, and may optionally comprise potassium. In one or more embodiments, the dehydrogenation catalysts may include catalysts disclosed in U.S. Pat. No. 8,669,406, which is incorporated herein by reference in its entirety, such as those including Ga, Cr, and/or Fe based catalysts. According to additional embodiments, Pt based catalysts may be utilized. In one or more embodiments, those catalysts disclosed in EP 0948475B1 and/or WO 2010/133565, which are each incorporated herein by reference in its entirety, may be utilized. Additional catalyst embodiments contemplated as suitable for use in the systems and methods described herein include those of U.S. Pat. No. 8,669,406, which is incorporated herein by reference in its entirety. Such catalysts may contain relatively low amounts of Cr, such as less than 6%, or approximately 1.5%. However, it should be understood that other suitable dehydrogenation catalysts may be utilized to perform the dehydrogenation reaction.

In one or more embodiments, the dehydrogenation catalyst may exhibit suitable stability when in the presence of steam. As is described herein, the combustion of hydrogen may form steam, which may be in direct contact with the dehydrogenation catalyst. It is contemplated that not all dehydrogenation catalysts are equally effective in steam environments. In one or more embodiments, dehydrogenation catalysts are utilized which maintain a substantial amount of their reactivity and/or selectivity for the dehydrogenation of light alkanes. For example, one or more of the dehydrogenation catalysts utilized in the presently disclosed systems and methods may not deteriorate in alkane conversion and/or selectivity for dehydrogenation more than 25%, more than 20%, more than 15%, more than 10%, more than 5%, or may even have improved alkane conversion and/or selectivity for dehydrogenation when in the presence of steam in amounts consistent with the operation of the presently disclosed systems. In some embodiments, the dehydrogenation catalyst may function with such conversion and/or selectivity when exposed to at least 10 mol. % water (such as from 10 mol. % to 50 mol. % water) for a period of up to, e.g., 120 seconds (the time which the catalyst may be exposed to such conditions, according to some embodiments of the presently disclosed system).

Suitable examples of dehydrogenation catalysts may be prepared such that it meets the Geldart A definition. In some embodiments, the dehydrogenation catalyst comprises gallium and platinum supported on alumina in the delta or theta phase, or in a mixture of delta plus theta phases, or theta plus alpha phases, or delta plus theta plus alpha phases, modified with silica, and having a surface area preferably less than about 100 square meters per gram (m²/g), as determined by the BET method. In other embodiments, the dehydrogenation catalyst comprises: from 0.1 to 34 wt. %, preferably 0.2 to 3.8 wt. %, gallium oxide (Ga₂O₃); from 1 to 300 parts per million (ppm), preferably 50 to 300 ppm, by weight platinum; from 0 to 5 wt. %, preferably 0.01 to 1 wt. %, of an alkaline and/or earth-alkaline such as potassium; from 0.08 to 3 wt. % silica; the balance to 100 wt. % being alumina.

In one or more embodiments, heat may be gained or lost through the dehydrogenation reaction, the re-oxidation of the oxygen-diminished oxygen carrier material, and the reduction of the oxygen-rich oxygen carrier material may create or use heat (i.e., be exothermic or endothermic). In one or more embodiments, the contacting of the hydrocarbon feed with the dehydrogenation catalyst may be endothermic and results in a dehydrogenation heat loss. In some embodiments, the contacting of the hydrogen with the oxygen-rich oxygen carrier material may be exothermic and results in a combustion heat gain. The re-oxidizing of the oxygen-diminished oxygen carrier material may be exothermic and results in an oxygenation heat gain. As such, by incorporating hydrogen combustion during catalytic dehydrogenation, in some embodiments, enough heat may be generated during the re-oxidation of the oxygen-diminished oxygen carrier material to act as a source of heat for the alkane to olefin reaction. As such, embodiments of the disclosed process may allow for higher alkane conversion while reducing or eliminating needs for fuel gas, as required for conventional cracking because the heat gained throughout the process by the re-oxidizing of the oxygen carrier material, the combustion of hydrogen, or both may produce the amount of heat required for the alkanes or alkyl aromatics to olefins reaction.

As presently described, the “dehydrogenation heat loss” refers to the amount of heat lost by the dehydrogenation of the feed alkanes, the “combustion heat gain” refers to the amount of heat created by the combustion of the hydrogen, and the “oxygenation heat gain” refers to the amount of heat created by the oxidation of the oxygen-diminished oxygen carrier material. In one or more embodiments, the combustion heat gain may contribute heat to the system that account for at least a portion of the dehydrogenation heat loss. In additional embodiments, supplemental fuel 172 may be combusted to heat one or more of the dehydrogenation catalyst or the oxygen carrier material. The supplemental fuel 172 may make up for any shortcoming in heat created by the combustion of the hydrogen or the oxygenation of the oxygen carrier material. However, it should be understood that in the embodiments disclosed, the amount of necessary supplemental fuel 172 may be substantially less than would be necessary in a system that did not incorporate an oxygen carrier material.

The production of olefinic compounds by conventional dehydrogenation processes (e.g., those that do not incorporate hydrogen combustion) may be relatively expensive due to the high heat loads needed for the endothermic dehydrogenation reaction and/or the downstream separation steps sometimes needed to separate the unreacted alkane or alkyl aromatic and remove hydrogen that is produced in the dehydrogenation reaction. Regarding reduced heat input, catalytic dehydrogenation processes are generally endothermic and require heat. However, the exothermic combustion of hydrogen can somewhat counterbalance that heat input requirement. Additionally, the oxygen carrier material, once diminished in oxygen content following the combustion, may be regenerated to regain its oxygen, which may be exothermic. This exothermic regeneration step may further counterbalance the heat input requirement to maintain the dehydrogenation reaction. In some embodiments, the heat produced by the oxygen carrier regeneration and combustion reaction may completely cover the heat needed for the endothermic dehydrogenation reaction and other heat demands such as heating the feed gases (air, hydrocarbon, etc.) or balancing heat losses, or at least reduce any supplemental fuel 172 needs of the system. For additional generalized information on dehydrogenation reactions that incorporate hydrogen combustion, those skilled in the art are referred to, for example, US Patent Publication 2021/0292259 A1, the teachings of which are incorporated herein by reference in their entirety.

Referring still to FIG. 1, during operation of the dehydrogenation reactor 110 of the reactor system 100, the feed stream 102 may enter a riser within the dehydrogenation reactor 110, and the product stream 114 may exit the reactor system 100 via stream 114. According to one or more embodiments, the reactor system 100 may be operated by feeding a chemical feed (e.g., in a feed stream 102 such as feed stream 102) into the dehydrogenation reactor 110.

According to one or more embodiments, a particulate solid 180 may be fed into the dehydrogenation reactor 110 via stream 152. The particulate solid 180 may include an oxygen-rich oxygen-carrier material. The particulate solid 180 may also include a dehydrogenation catalyst. The feed stream 102 may contact the particulate solid 180 in the dehydrogenation reactor 110. Each of the feed stream 102 and the particulate solid 180 may flow upwardly into and through the dehydrogenation reactor 110 to produce one or more products, an oxygen-diminished oxygen-carrier material, and hydrogen. In the dehydrogenation reactor 110 the one or more hydrocarbons in the feed stream 102 may be dehydrogenated to form one or more products and hydrogen. Additionally, within the dehydrogenation reactor 110, the hydrogen may be contacted with the oxygen-rich oxygen-carrier material in the dehydrogenation reactor 110. The oxygen-rich oxygen-carrier material may be reducible. The contacting of the oxygen-rich oxygen-carrier material with the hydrogen may combust the hydrogen and reduce the oxygen-carrier material to form an oxygen-diminished oxygen-carrier material and water.

In one or more embodiments, one or more products may exit the dehydrogenation reactor 110 via product stream 114. Stream 114 may be further processed such as by one or more subsequent separation steps or further reacted. It is contemplated that stream 114 may be utilized as a feed for another reactor system or sold as a chemical product. In embodiments, the one or more products in stream 114 may be mixed with water produced from reacting the hydrogen produced during the dehydrogenation of the one or more hydrocarbons and oxygen from the oxygen-carrier material. In some embodiments, the water may be removed from stream 114 and the one or more products utilizing a condenser.

As described above, in some embodiments, stream 114 may comprise one or more products. In one or more embodiments, the one or more products may comprise one or more olefinic compounds. As used herein, the term “olefinic compounds” refers to hydrocarbons having one or more carbon-carbon double bonds apart from the formal double bonds in aromatic compounds. For example, ethylene and styrene are olefinic compounds, but ethylbenzene would not be an olefinic compound as the only double bonds present in ethylbenzene are formal double bonds present as part of the aromatic structure. In one or more embodiments, the one or more olefinic compounds may comprise one or more of ethylene, propylene, butylene, or styrene. In some embodiments, the product stream 114 may comprise at least 50 wt. %, at least 60 wt. %, at least 70 wt. %, at least 80 wt. %, at least 90 wt. %, at least 95 wt. % or even at least 99 wt. % of ethylene. In additional embodiments, stream 114 may comprise at least 50 wt. %, at least 60 wt. %, at least 70 wt. %, at least 80 wt. %, at least 90 wt. %, at least 95 wt. % or even at least 99 wt. % of propylene. In additional embodiments, stream 114 may comprise at least 50 wt. %, at least 60 wt. %, at least 70 wt. %, at least 80 wt. %, at least 90 wt. %, at least 95 wt. % or even at least 99 wt. % of butylene. In additional embodiments, stream 114 may comprise at least 50 wt. %, at least 60 wt. %, at least 70 wt. %, at least 80 wt. %, at least 90 wt. %, at least 95 wt. % or even at least 99 wt. % of styrene. In additional embodiments, stream 114 may comprise at least 50 wt. %, at least 60 wt. %, at least 70 wt. %, at least 80 wt. %, at least 90 wt. %, at least 95 wt. % or even at least 99 wt. % of the sum of one or more of ethylene, propylene, butylene, and styrene.

In some embodiments, the one or more products and the particulate solid 180 may be passed to a separation device in a separation section within the dehydrogenation reactor 110. The particulate solid 180 may be separated from the one or more products in a separation device (not depicted in FIG. 1) within the dehydrogenation reactor 110. The one or more products may then be transported out of the separation section of the dehydrogenation reactor 110. For example, the separated vapors may be removed from the dehydrogenation reactor 110 via a pipe at a gas outlet port of the separation section within the dehydrogenation reactor 110. According to one or more embodiments, the separation device may be a cyclonic separation system. The cyclonic separation system may include two or more stages of cyclonic separation.

In one or more embodiments, the dehydrogenation reactor 110 may operate with a residence time of the vapor in the dehydrogenation reactor 110 of less than 10 seconds (such less than 9 seconds, less than 8 seconds, less than 7 seconds, less than 6 seconds, less than 5 seconds, less than 4 seconds, or even less than 3 seconds.

In one or more embodiments, the dehydrogenation reactor 110 may operate at a temperature of greater than or equal to 550° C. and less than or equal to 800° C. In some embodiments, the temperature in the dehydrogenation reactor 110 may be from 550° C. or 600° C. to 770° C. In other embodiments, the temperature in the dehydrogenation reactor 110 may be from 700° C. to 750° C. Without being bound by any particular theory, it is believed that too low of temperature (e.g., 550° C. or less) may limit the maximum conversion of the hydrocarbon due to equilibrium constraints as well as lowers the rate of dehydrogenation by the thermal and catalytic component. Too low of temperatures may also result in a slow rate of oxygen release from the oxygen-carrier material and low hydrogen combustion. On the other hand, high temperatures (e.g., greater than 800° C.) may result in thermal degradation of the products produced and may result in a lower product selectivity than is economically feasible. In some embodiments, the primary feed component(s) may be propane, ethylbenzene, and/or butane, and the dehydrogenation reactor 110 may operate at a temperature of greater than 600° C. In additional embodiments, the primary feed component may be ethane, and the dehydrogenation reactor 110 may operate at a temperature of at least 625° C.

In some embodiments, the dehydrogenation reactor 110 may operate at a pressure of at least atmospheric pressure (about 14.7 psia). In some embodiments, the dehydrogenation reactor 110 may operate at a pressure of about 500 psia. In other embodiments, the dehydrogenation reactor 110 may operate at a pressure from about 4 psia to about 160 psia, from about 20 psia to about 100 psia, or from about 30 psia to about 80 psia. In some embodiments, the regeneration unit 150 may operate with a pressure of within 30 psi of the dehydrogenation reactor 110.

The residence time of the particulate in the dehydrogenation reactor 110 may typically vary from 0.5 seconds (sec) to 360 sec. In other embodiments, the residence time of the particulate solid 180 may be from about 0.5 sec to about 200 sec, from about 0.5 sec to about 100 sec, from about 0.5 sec to about 50 sec, or about 0.5 sec to about 20 sec.

In additional embodiments, the ratio of the particulate solid 180 to the feed stream 102 in the dehydrogenation reactor 110 may range from 5 to 150 on a weight to weight (w/w) basis. In some embodiments, the ratio may range from 10 to 40, such as from 12 to 36, or from 12 to 24.

In additional embodiments, the flux of the particulate solid 180 may be from 1 pound per square foot-second (lb/ft²-s) (about 4.89 kg/m²-s) to 300 lb/ft²-s (to about 97.7 kg/m²-s), such as from 1-20 lb/ft²-s, in the upstream reactor section, and from 1 lb/ft²-s (about 48.9 kg/m²-s) to 300 lb/ft²-s (about 489 kg/m²-s), such as from 10-100 lb/ft²-s, in the downstream reactor section.

In one or more embodiments, the particulate solid 180 may be capable of fluidization. In some embodiments, the particulate solid 180 may exhibit properties known in the industry as “Geldart A” or “Geldart B” properties. Particles may be classified as “Group A” or “Group B” according to D. Geldart, Gas Fluidization Technology, John Wiley & Sons (New York, 1986), 34-37; and D. Geldart, “Types of Gas Fluidization,” Powder Technol. 7 (1973) 285-292, which are incorporated herein by reference in their entireties.

Group A is understood by those skilled in the art as representing an aeratable powder, having a bubble-free range of fluidization; a high bed expansion; a slow and linear deaeration rate; bubble properties that may include a predominance of splitting/recoalescing bubbles, with a maximum bubble size and large wake; high levels of solids mixing and gas backmixing, assuming equal U-Umf (U is the velocity of the carrier gas, and Umf is the minimum fluidization velocity, typically though not necessarily measured in meters per second, m/s, i.e., there is excess gas velocity); axisymmetric slug properties; and no spouting, except in very shallow beds. The properties listed tend to improve as the mean particle size decreases, assuming equal cfp; or as the <45 micrometers (μm) proportion is increased; or as pressure, temperature, viscosity, and density of the gas increase. In general, the particles may exhibit a small mean particle size and/or low particle density (<1.4 grams per cubic centimeter, g/cm³), fluidize easily, with smooth fluidization at low gas velocities, and may exhibit controlled bubbling with small bubbles at higher gas velocities.

Group B is understood by those skilled in the art as representing a “sand-like” powder that starts bubbling at Umf; that exhibits moderate bed expansion; a fast deaeration; no limits on bubble size; moderate levels of solids mixing and gas backmixing, assuming equal U-Umf; both axisymmetric and asymmetric slugs; and spouting in only shallow beds. These properties tend to improve as mean particle size decreases, but particle size distribution and, with some uncertainty, pressure, temperature, viscosity, or density of gas seem to do little to improve them. In general, most of the particles having a particle size (cfp) of 40 μm<cfp<500 μm when the density (pp) is 1.4<pp<4 g/cm³, and preferably 60 μm<cfp<500 μm when the density (pp) is 4 g/cm³ and 250 μm<cfp<100 μm when the density (pp) is 1 g/cm³.

Referring still to FIG. 1, in one or more embodiments, the particulate solid 180, and the gas products may be separated within the dehydrogenation reactor 110 by high efficiency cyclones in a stripping section of the reactor system 100 (not pictured in FIG. 1). The stripping section may be within the reactor 110 or may be a separate vessel. In embodiments described, the particulate solid 180 may be passed to the regeneration unit 150 via stream 112. In some embodiments, the dehydrogenation catalyst, the oxygen-carrier material, or both may be stripped in the stripping section with a displacement gas such as nitrogen, steam, methane, natural gas or other suitable gas before being sent to a regeneration unit 150. In some embodiments, a portion of the particulate solid 180 may be passed from the stripping section via stream 116 to stream 152 to be used again in the reactor 110 without first passing through the regeneration unit 150. Without being bound by theory it is believed that by recycling a portion of the particulate solid 180 back to the reactor 110 the oxygen-content of the oxygen-carrier material may be better controlled as the oxygen-carrier material may be given more time to react off its oxygen so that the oxygen-carrier material may supply more oxygen to the reactor. It is also believed that recycling the particulate solid 180 may allow for improved temperature control of the reactor 110 because recycling the particulate solid 180 may improve control of the temperature of the materials entering the reactor 110 improving the temperature profile of the reactor 110.

In some embodiments, the dehydrogenation catalyst of the particulate solid 180 may be slightly deactivated after contacting the feed stream 102. In other embodiments, the dehydrogenation catalyst of the particulate solid 180 may still be suitable for reaction in the dehydrogenation reactor 110. As used herein, “deactivated” may refer to a catalyst that is contaminated with a substance such as coke, is cooler in temperature than needed to promote reaction of the feed, or may refer to an oxygen-carrier material that is deficient of oxygen. In some embodiments, a contaminate, such as coke, may be deposited on the particulate solid 180 that is passed from the dehydrogenation reactor 110 to the regeneration unit 150.

The particulate solid 180 may enter the regeneration unit 150 via stream 112. The particulate solid 180 may then enter the gas/solids separator 158 within the regeneration unit 150. Exiting the separator 158, the particulate solid 180 may pass through an air zone 164 and past the first gas inlet 160 through which the oxygen-containing gas 162 may enter into the regeneration unit 150. The particulate solid 180 may then pass through a fuel zone 174 and past the second gas inlet 170 through which the fuel 172 may enter into the regeneration unit 150. The particulate solid 180 may then exit the regeneration unit 150 via stream 152 and be passed back to the dehydrogenation reactor 110.

Regeneration may remove contaminates, such as coke, raise the temperature of the particulate solid 180, increase the oxygen-content of the oxygen-carrier material of the particulate solid 180, or combinations thereof. In some embodiments, the coke on the particulate solid 180 may be removed by combustion in an oxygen-containing environment in the regeneration unit 150. In further embodiments, the particulate solid 180 may be heated by a fuel 172 to a target temperature. The particulate solid 180 may then circulate back to the dehydrogenation reactor 110, carrying the necessary heat for the dehydrogenation reaction. For additional generalized information on dehydrogenations carried out in fluidized beds, those skilled in the art are referred to, for example, US Patent Publication 2005/0177016 A1; International Patent Publication WO 2005/077867 A1(corresponding to US Patent Publication 2008/0194891 A1); and International Patent Publication WO 20107591 A1.

The oxygen-carrier may be oxidized or reduced as it is utilized in a dehydrogenation process. Different oxidation states of the oxygen-carrier may behave differently within the dehydrogenation process. For example, an oxygen-carrier with a lower oxidation state may not as effectively combust hydrogen within the dehydrogenation reaction as an oxygen-carrier with a comparatively higher oxidation state. Conversely, an oxygen-carrier with a comparatively higher oxidation state may be more likely to oxidize hydrocarbons during the dehydrogenation reaction when compared to an oxygen-carrier with a comparatively lower oxidation state potentially harming the production of olefins from the process. Passing the oxygen-carrier to and through the regeneration unit 150 may help control the oxidation state of the oxygen-carrier material.

In some embodiments, the oxygen-diminished oxygen carrier material of the particulate solid 180 may be re-oxidized to an oxidation state higher than the oxidation state of the oxygen-diminished oxygen carrier material by combustion in an oxygen-containing environment in the regeneration unit 150. In some embodiments, the oxygen-containing environment may be air. In some embodiments of forming the oxygen-rich oxygen carrier material, the oxygen-diminished oxygen carrier material may be restored to its original oxidation state. In some embodiments, the oxygen-diminished oxygen carrier material may have an oxidation state of +2, +3, or +4. The oxygen-rich oxygen carrier material may then circulate back to the dehydrogenation reactor 110, carrying the necessary heat for the dehydrogenation reaction. In other embodiments, nitrogen or steam may also be used to convey the oxygen-rich oxygen carrier material to the dehydrogenation reactor 110. The resulting gas stream 154 from the regeneration unit 150 may consist of air depleted of or containing a lower concentration of O₂.

In one or more embodiments, a supplemental fuel 172 may be combusted in the regeneration unit 150 to produce heat and increase the temperature of the particulate solid 180. The heat produced by the oxidizing of the oxygen-diminished oxygen carrier material and the combusting of the supplemental fuel 172 may be sufficient to maintain the temperature of the dehydrogenation reactor 110 at a desired temperature. The desired temperature may depend upon the minimum temperature needed for operation of the dehydrogenation reactor 110, since the particulate solid 180 may enter the dehydrogenation reactor 110 and impart its temperature to the dehydrogenation reactor 110.

In one or more embodiments, the regeneration unit 150 may operate at a temperature of 650° C., or even 700° C., to 900° C., such as 725° C. to 875° C., or 750° C. to 850° C. Generally, the regeneration unit 150 may have a temperature of at least 50° C. greater than that of the dehydrogenation reactor 110. Such a temperature range may be utilized so that the temperature of the dehydrogenation reactor 110 may be maintained with a limited amount of dehydrogenation catalyst and/or oxygen carrier material. Additionally, such temperatures may be needed to activate the dehydrogenation catalyst if one is utilized.

The residence time of the particulate solid 180 in the regeneration unit 150 may typically vary from 0.5 seconds (sec) to 240 sec. In other embodiments, the residence time of the particulate solid 180 may be from about 0.5 sec to about 200 sec, from about 0.5 sec to about 100 sec, from about 0.5 sec to about 50 sec, or about 0.5 sec to about 20 sec.

Referring still to FIG. 1, in one or more embodiments, the particulate solid 180 may be passed from the dehydrogenation reactor 110 to the regeneration unit 150 via stream 112. In the regeneration unit 150 the particulate solid 180 may be passed through a gas/solids separator 158. According to one or more embodiments, the separator 158 may be a riser termination system which may include a cyclonic separation system. In embodiments, where the riser termination system includes a cyclonic separation system some of the particulate solid 180 may be passed out of the riser termination system without first passing through the cyclonic separation system. The cyclonic separation system may include two or more stages of cyclonic separation. In embodiments where the separator 158 comprises more than one cyclonic separation stages, the first separation device into which the fluidized stream enters is referred to a primary cyclonic separation device. The fluidized effluent from the primary cyclonic separation device may enter into a secondary cyclonic separation device for further separation. Primary cyclonic separation devices may include, for example, primary cyclones, and systems commercially available under the names VSS (commercially available from UOP), LD2 (commercially available from Stone and Webster), and RS2 (commercially available from Stone and Webster). Primary cyclones are described, for example, in U.S. Pat. Nos. 4,579,716; 5,190,650; and 5,275,641, which are each incorporated by reference in their entirety herein. In some separation systems utilizing primary cyclones as the primary cyclonic separation device, one or more set of additional cyclones, e.g. secondary cyclones and tertiary cyclones, are employed for further separation of the particulate solid from the product gas. It should be understood that any primary cyclonic separation device may be used in embodiments of the invention. The particulate solid 180 may be separated from exhaust gases such as flue gas. The gases separated from the particulate solid 180 may combine with gases from the regeneration unit 150 and exit the regeneration unit 150 via an exhaust stream 154.

According to embodiments, the particulate solid 180 may move in a generally downward direction through the regeneration unit 150. As used in the present disclosure the term “generally downward direction” means that the average velocity of the particulate solid 180 is in the downward direction, where the downward direction is with the pull of gravity. As it is an average, the velocity of individual particles of the particulate solid 180 may have a distribution and may not be equal to the average, but taken as a whole the velocity of the particulate solid 180 will average out to be generally downward. According to embodiments, gases within the regeneration unit 150 may move in a generally upwards direction through the regeneration unit 150. As used in the present disclosure the term “generally upward direction” means that the average velocity of the gases is in the upward direction, where the upward direction is against the pull of gravity. As it is an average, the velocity of the gas molecules within the regeneration unit 150 may have a distribution and may not be equal to the average, but taken as a whole the velocity of the gases will average out to be generally upward. According to embodiments, the particulate solid 180 and the gases may move in a countercurrent flow pattern through the regeneration unit 150.

In one or more embodiments, the regeneration unit 150 may comprise a first gas inlet 160 and a second gas inlet 170 beneath the first gas inlet 160. As used herein, “gas inlet” refers to any component operable to inject a gas into the regeneration unit 150. For example, a gas inlet may be a blower or distributor. Additionally, other suitable gas suppliers are contemplated herein, as would be known by those skilled in the art.

According to embodiments, an oxygen-containing gas 162 may enter the regeneration unit 150 via the first gas inlet 160. In some embodiments, the oxygen-containing gas 162 may be air, enriched air, air mixed with steam, or flue gas. Enriched air is air with added oxygen gas. In some embodiments, the oxygen-containing gas 162 may include at least 28 mol. % oxygen. In other embodiments, the oxygen-containing gas 162 may include from about 2 mol. % to about 28 mol. % oxygen, from about 2 mol. % to about 25 mol. %, from about 2 mol. % to about 20 mol. %, from about 2 mol. % to about 15 mol. %, from about 2 mol. % to about 10 mol. %, from about 2 mol. % to about 5 mol. %, from about 5 mol. % to about 28 mol. %, from about 5 mol. % to about 25 mol. %, from about 5 mol. % to about 20 mol. %, from about 5 mol. % to about 15 mol. %, from about 5 mol. % to about 10 mol. %, from about 10 mol. % to about 28 mol. %, from about 10 mol. % to about 25 mol. %, from about 10 mol. % to about 20 mol. %, from about 10 mol. % to about 15 mol. %, from about 15 mol. % to about 28 mol. %, from about 15 mol. % to about 25 mol. %, from about 15 mol. % to about 20 mol. %, from about 20 mol. % to about 28 mol. %, from about 20 mol. % to about 25 mol. %, or from about 25 mol. % to about 28 mol. %.

In embodiments, the oxygen-containing gas 162 may enter the regeneration unit 150 in a generally upwards direction. In some embodiments, the oxygen-containing gas 162 may initially enter the regeneration unit 150 in a generally downwards direction before following the flow of other gases within the regeneration unit 150 generally upwards. In some embodiments, the area above the first gas inlet 160 may be an air zone 164 as the directional flow of the oxygen-containing gas 162 into the regeneration unit 150 may cause the concentration of oxygen in the area above the first gas inlet 160 to be higher than other areas of the regeneration unit 150. In one or more embodiments, the concentration of oxygen in the air zone 164 may be greater than 25 mol. %. In other embodiments, the concentration of oxygen in the air zone 164 may be from about 4 mol. % to about 28 mol. % oxygen, from about 4 mol. % to about 21 mol. %, from about 4 mol. % to about 10 mol. %, from about 10 mol. % to about 28 mol. %, from about 10 mol. % to about 21 mol. %, or from about 21 mol. % to about 28 mol. % oxygen. The name air is utilized here to simply show that oxygen is present since air is often the oxygen-containing gas utilized at least cost.

In one or more embodiments, a fuel 172 may enter the regeneration unit 150 via the second gas inlet 170. In some embodiments, the fuel 172 may comprise hydrogen, methane, ethane, propane, natural gas, or combinations thereof. According to embodiments, the fuel 172 may enter the regeneration unit 150 in a generally upwards direction. In some embodiments, the fuel 172 may enter the regeneration unit initially in a generally downwards direction before following the flow of other gases within the regeneration unit 150 generally upwards. In embodiments, at least a portion of the fuel 172 in the area above the second gas inlet 170 and beneath the first gas inlet 160 may react with oxygen from the oxygen-carrier material of the particulate solid 180 to produce heat, increase the temperature of the particulate solid 180, and reduce the oxygen-content. In embodiments, the area above the second gas inlet 170 and beneath the first gas inlet 160 may be a fuel zone 174, as the directional flow of the fuel 172 into the regeneration unit 150 may cause the concentration of fuel 172 in the area above the second gas inlet 170 and beneath the first gas inlet 160 to be higher than the concentration of fuel 172 in the area above the first gas inlet 160 or in the area below the second gas inlet 170.

In some embodiments, the concentration of fuel 172 in the fuel zone 174 may be greater than 20 mol. %, greater than 50 mol. %, or even greater than 90 mol. %. In some embodiments the concentration of fuel 172 in the fuel zone 174 may be from about 40 mol. % to about 100 mol. %, from about 40 mol. % to about 90 mol. %, from about 40 mol. % to about 80 mol. %, from about 40 mol. % to about 70 mol. %, from about 40 mol. % to about 60 mol. %, from about 40 mol. % to about 50 mol. %, from about 50 mol. % to about 100 mol. %, from about 50 mol. % to about 90 mol. %, from about 50 mol. % to about 80 mol. %, from about 50 mol. % to about 70 mol. %, from about 50 mol. % to about 60 mol. %, from about 60 mol. % to about 100 mol. %, from about 60 mol. % to about 90 mol. %, from about 60 mol. % to about 80 mol. %, from about 60 mol. % to about 70 mol. %, from about 70 mol. % to about 100 mol. %, from about 70 mol. % to about 90 mol. %, from about 70 mol. % to about 80 mol. %, from about 80 mol. % to about 100 mol. %, from about 80 mol. % to about 90 mol. %, or from about 90 mol. % to about 100 mol. %.

In embodiments, at least a portion of the oxygen-diminished oxygen-carrier material of the particulate solid 180 may react with oxygen from the oxygen-containing gas 162 in the air zone 164 and the oxygen content of at least a portion of the oxygen-carrier material of the particulate solid 180 may increase to form the oxygen-rich oxygen-carrier material of the particulate solid 180. In some embodiments, at least a portion of the fuel 172 may not be combusted in the fuel zone 174 and may travel upwards through the regeneration unit 150 into the air zone 164. In some embodiments, at least a portion of the fuel 172 may react with oxygen from the oxygen-containing gas 162 in the area above the first gas inlet 162. In some embodiments, any fuel 172 remaining in the regeneration unit 150 after fuel 172 combustion in the fuel zone 174 may be combusted in the air zone 164, such that most of the fuel 172 that enters regeneration unit 150 through the second gas inlet 170 is combusted within the regeneration unit 150. It may be desirable to ensure most of the fuel 172 is combusted within the regeneration unit 150 to, for example, sufficiently heat the particulate solid 180 using a minimum amount of fuel 172 or to ensure only a minimal amount of fuel 172 exits the regeneration unit 150 to downstream processes which may improve the safety of those processes. In one or more embodiments, at least a portion of the particulate solid 180 may be passed from the regeneration unit 150 back to the dehydrogenation reactor 110 via stream 152.

Without being bound by theory, it is believed that by exposing the oxygen-carrier material to a fuel 172 in an area of the regeneration unit 150 after exposing the oxygen-carrier material to an oxygen-containing gas 162 the oxygen content of at least a portion of the oxygen-carrier material may be reduced as oxygen from the oxygen-carrier material reacts with the fuel 172. The oxygen-carrier material may then exit the regeneration unit 150 without replacing the oxygen released during fuel 172 combustion returning to the dehydrogenation reactor 110 in a pre-reduced state. Some oxygen-carrier materials may have the desired activity and selectivity for certain reaction conditions in a reduced state that is below the maximum oxidation level of the material. It may therefore be desired to pre-reduce the material before it exits the regeneration unit 150. For example, exposing the oxygen-carrier material to a fuel 172 below an oxygen-containing gas 162 in a counter-current flow pattern regeneration unit 150 may allow the oxygen-carrier material to be sufficiently pre-reduced before exiting the regeneration unit 150, such that further reduction may not be required.

It is also believed that pre-reduction using a supplemental fuel 172 in the regeneration unit 150 may utilize enough fuel 172 combustion to sufficiently heat the particulate solid 180 for use in the dehydrogenation reaction. Further, pre-reduction using a fuel 172 may produce exhaust gases such as carbon dioxide and water. It may be beneficial to produce these gases in the regeneration unit 150 to allow them to be exhausted with the other combustion gases produced in the regeneration from, for example, the combustion of coke on the particulate solid 180.

Now referring to reactor system 200 in FIG. 2, a reactor system 200 according to additional embodiments of the present disclosure is shown. The particulate solid 180 may be first passed from the dehydrogenation reactor 110 to a pre-oxidation unit 210 via stream 112 before being to the regeneration unit 150. An oxygen-containing gas 212 may enter the pre-oxidation unit 210 and the particulate solid 180 may be exposed to the oxygen-containing gas 212. The oxygen-containing gas 212 and the particulate solid 180 may flow generally co-currently in an upward direction through the pre-oxidation unit 210 and may exit the pre-oxidation unit 210 via stream 222 and may then be passed to the regeneration unit 150. The particulate solid 180 may flow generally in a downward direction through the regeneration unit 150 first through the air zone 164, then past a third gas inlet 140 beneath the first gas inlet 160 and above the second gas inlet 170 through which a stripping gas 142 may enter the regeneration unit 150. The particulate solid 180 may flow through the fuel zone 174 and then past a fourth gas inlet 130 beneath the second gas inlet 170 through which a stripping gas 132 may enter into the regeneration unit 150. The particulate solid 180 may then exit the regeneration unit 150 via stream 152 and be passed back to the dehydrogenation reactor 110. The pre-oxidation unit 210 and the third gas inlet 140 are optional additions to the reactor system 200. In one or more embodiments, the reactor system 200 may comprise the pre-oxidation unit 210, but not the third gas inlet 140 or the fourth gas inlet 130. In some embodiments, the reactor system 200 may comprise the third gas inlet 140, but not the pre-oxidation unit 210 or the fourth gas inlet 130. In some embodiments, the reactor system 200 may comprise the fourth gas inlet 130, but not the pre-oxidation unit 210 or the third gas inlet 140. In some embodiments, the reactor system 200 may comprise the pre-oxidation unit 210 and the third gas inlet 140, but not the fourth gas inlet 130. In some embodiments, the reactor system 200 may comprise the pre-oxidation unit 210 and the fourth gas inlet 130, but not the third gas inlet 140. In some embodiments, the reactor system 200 may comprise the third gas inlet 140 and the fourth gas inlet 130, but not the pre-oxidation unit 210.

Now referring to FIG. 2, in one or more embodiments, the particulate solid 180 may be passed from the dehydrogenation reactor 110 to a pre-oxidation unit 210 before being passed to the regeneration unit 150. In one or more embodiments, an oxygen-containing gas 212 may enter the pre-oxidation unit 210. In some embodiments, the oxygen-containing gas 212 may be air, enriched air, air mixed with steam, or flue gas. Enriched air is air with added oxygen gas. In some embodiments, the oxygen-containing gas 212 may include at least 28 mol. % oxygen. In other embodiments, the oxygen-containing gas 212 may include from about 2 mol. % to about 28 mol. % oxygen, from about 2 mol. % to about 25 mol. %, from about 2 mol. % to about 20 mol. %, from about 2 mol. % to about 15 mol. %, from about 2 mol. % to about 10 mol. %, from about 2 mol. % to about 5 mol. %, from about 5 mol. % to about 28 mol. %, from about 5 mol. % to about 25 mol. %, from about 5 mol. % to about 20 mol. %, from about 5 mol. % to about 15 mol. %, from about 5 mol. % to about 10 mol. %, from about 10 mol. % to about 28 mol. %, from about 10 mol. % to about 25 mol. %, from about 10 mol. % to about 20 mol. %, from about 10 mol. % to about 15 mol. %, from about 15 mol. % to about 28 mol. %, from about 15 mol. % to about 25 mol. %, from about 15 mol. % to about 20 mol. %, from about 20 mol. % to about 28 mol. %, from about 20 mol. % to about 25 mol. %, or from about 25 mol. % to about 28 mol. %.

In embodiments, the oxygen-carrier material of the particulate solid 180 may be exposed to the oxygen-containing gas 212 in the pre-oxidation unit 210. In some embodiments, the content of oxygen in at least a portion of the oxygen-carrier material of the particulate solid 180 may be increased in the pre-oxidation unit 210. In some embodiments, the oxygen-containing gas 162 and the particulate solid 180 may flow generally co-currently in an upward direction through the pre-oxidation unit 210. As used herein, “generally co-currently” means that the average velocities of the particulate solid 180 and the oxygen-containing gas 162 are in the same direction. As it is an average, the velocity of individual particles of the particulate solid 180 or the individual gas molecules of the oxygen-containing gas 162 may have a distribution and may not be equal to the average, but taken as a whole the velocity of both components will average out to be generally in the same direction. In some embodiments, coke that may form on the particulate solid 180 in the dehydrogenation reactor 110 may be combusted in the pre-oxidation unit 210 heating at least a portion of the particulate solid 180. In some embodiments, carryover fuel 172 from the regeneration unit 150, the reactor 110 or both may be reacted in the pre-oxidation unit 210 heating at least a portion of the particulate solid 180.

In one or more embodiments, the particulate solid 180 may be passed from the pre-oxidation unit 210 to the regeneration unit 150 via a conduit 222. In some embodiments, the particulate solid 180 may be mixed with gases, such as the oxygen-containing gas 212 from the pre-oxidation unit 210, combustion gases from the combustion of coke in the pre-oxidation unit 210, carry-over gases from the dehydrogenation reactor 110, or combinations thereof as it is passed to the regeneration unit 150. In one or more embodiments, the particulate solid 180 may be passed from the regeneration unit 150 back to the pre-oxidation unit 210 via stream 156, such that the particulate solid 180 may pass through the pre-oxidation unit 210 and the regeneration unit 150 at least one additional time.

In one or more embodiments, the particulate solid 180 may be passed from the regeneration unit 150 to the pre-oxidation unit 210. For example, the particulate solid 180 may be passed back to the pre-oxidation unit 210 to achieve a desired oxidation level of the oxygen-carrier material of the particulate solid 180 or to remove coke from the particulate solid 180. In some embodiments, the particulate solid 180 may be passed from the fuel zone 174 out of the regeneration unit 150 to the pre-oxidation unit 210 to be passed through the pre-oxidation unit 210 and the regeneration unit 150 again. In other embodiments, the particulate solid 180 may be passed from the air zone 164 out of the regeneration unit 150 to the pre-oxidation unit 210 to be passed through the pre-oxidation unit 210 and the regeneration unit 150 again. In further embodiments, the particulate solid 180 may be passed from the regeneration unit 150 to the pre-oxidation unit 210 from both the air zone 164 and the fuel zone 174.

Referring still to the reactor system 200 of FIG. 2, in one or more embodiments, the regeneration unit 150 may comprise a third gas inlet 140. In embodiments, a stripping gas 142 may enter the regeneration unit 150 through the third gas inlet 140. In embodiments, the third gas inlet 140 may be beneath the first gas inlet 160 and above the second gas inlet 170. In embodiments, the stripping gas 142 may exit the third gas inlet 140 in a generally upwards direction. In some embodiments the stripping gas 142 may exit the third gas inlet in an initially downwards direction before following the flow of the other gases within the regeneration unit 150 generally upwards. Accordingly, in embodiments, the area above the third gas inlet 140 and below the first gas inlet 160 may be a first strip zone 144, as the directional flow of the stripping gas 142 into the regeneration unit 150 may cause the concentration of stripping gas 132 to be higher in the area above the third gas inlet 140 and below the first gas inlet 160. In some embodiments, the stripping gas 142 may comprise nitrogen, steam, or combinations thereof. Without being bound by theory, it is believed that the use of a first strip zone 144 may limit or prevent gas-phase oxygen from the oxygen-containing gas 162 from entering the fuel zone 174. It is believed that gas-phase oxygen in the fuel zone 174 may oxidize the oxygen-carrier material or may limit the amount of oxygen from the oxygen-carrier material that is used to react the fuel 172, such that the reduction of the oxygen-carrier material in the fuel zone 174 is decreased.

In one or more embodiments, the regeneration unit 150 may comprise a fourth gas inlet 130. In embodiments, the fourth gas inlet 130 may be beneath the second gas inlet 170. In one or more embodiments, a stripping gas 132 may enter the regeneration unit 150 through the fourth gas inlet 130. In embodiments, the stripping gas 132 may exit the fourth gas inlet 130 in a generally upwards direction. Accordingly, in embodiments, the area above the fourth gas inlet 130 and below the second gas inlet 170 may be a second strip zone 134, as the directional flow of the stripping gas 132 into the regeneration unit 150 may cause the concentration of stripping gas 132 to be higher in the area above the fourth gas inlet 130 and below the second gas inlet 170. In some embodiments, the stripping gas 132 may comprise nitrogen, steam, or combinations thereof. Without being bound by theory, it is believed that the use of a second strip zone 134 beneath the fuel zone 174 may limit or prevent the carryover of fuel 172 from the fuel zone 174 to the reactor 110. Carryover fuel 172 entering the reactor 110 may prevent the reactor 110 from operating efficiently by, for example, combusting in the reactor 110 to raise the temperature in the reactor 110 or by combusting in the reactor 110 utilizing oxygen from the oxygen-carrier material limiting the amount of oxygen available to convert hydrogen to water in the reactor 110.

In one or more embodiments, the regeneration unit 150 may comprise both a third gas inlet 140 and a fourth gas inlet 130. In other embodiments the regeneration unit 150 may comprise a third gas inlet 140, but not a fourth gas inlet 130. In other embodiments, the regeneration unit 150 may comprise a fourth gas inlet 130, but not a third gas inlet 140. Accordingly in one or more embodiments the regeneration unit 150 may comprise a first strip zone 144, but not a second strip zone 134, a second strip zone 134, but not a first strip zone 144, or both a first strip zone 144 and a second strip zone 134.

In one or more embodiments, at least a portion of the particulate solid 180 may be withdrawn from the regeneration unit 150 and passed through at least a portion of the regeneration unit 150 a second time before being passed to the dehydrogenation reactor 110. Accordingly, in some embodiments, at least a portion of the particulate solid 180 may be withdrawn from the area above the fuel zone 174, withdrawn from the area above the first strip zone 144, withdrawn from the area beneath the second strip zone 134, withdrawn from the air zone 164, or withdrawn from combinations thereof. In embodiments, the withdrawn particulate solid 180 may be added back to the regeneration unit 150 at any point above where the withdrawn particulate solid 180 was withdrawn from. For example, if the portion of the particulate solid 180 was withdrawn from the fuel zone 174 it may be passed back into the regeneration unit 150 anywhere above the fuel zone 174, such as the air zone 164, the first strip zone 144, or both.

In a first aspect of the present disclosure, hydrocarbons may be dehydrogenated by a method comprising contacting a feed stream comprising one or more hydrocarbons with a particulate solid in a dehydrogenation reactor. The particulate solid comprises an oxygen-carrier material. In the dehydrogenation reactor one or more hydrocarbons are dehydrogenated to form hydrogen and one or more products. At least a portion of the hydrogen is reacted with oxygen from the oxygen-carrier material to form water and reduce the oxygen content in the oxygen-carrier material. The process further comprises passing the particulate solid from the dehydrogenation reactor to a regeneration unit. The particulate solid moves in a generally downward direction through the regeneration unit and gases move in a generally upwards direction through the regeneration unit, such that the particulate solid and the gases move in a countercurrent flow pattern through the regeneration unit. The regeneration unit comprises a first gas inlet and a second gas inlet. The second gas inlet is beneath the first gas inlet. An oxygen-containing gas enters into the regeneration unit through the first gas inlet. A fuel enters into the regeneration unit through the second gas inlet. In an area of the regeneration unit above the second gas inlet and beneath the first gas inlet at least a portion of the fuel is reacted with oxygen from the oxygen-carrier material of the particulate solid. The method further comprises passing at least a portion of the particulate solid from the regeneration unit to the dehydrogenation unit.

A second aspect of the present disclosure may include the first aspect, where in an area of the regeneration unit above the first gas inlet, the oxygen content in at least a portion of the oxygen-carrier material of the particulate solid increases.

A third aspect of the present disclosure includes any previous aspect or combination of aspects, where at least a portion of the fuel passes to the area of the regeneration unit above the first gas inlet, and in the area of the regeneration unit above the first gas inlet at least a portion of the fuel is reacted with oxygen from the oxygen-containing gas.

A fourth aspect of the present disclosure includes any previous aspect or combination of aspects, where the one or more hydrocarbons comprise an alkyl moiety and the one or more products comprise one or more olefinic compounds.

A fifth aspect of the present disclosure includes any previous aspect or combination of aspects, where the one or more hydrocarbons comprise ethane and the one or more products comprise ethylene.

A sixth aspect of the present disclosure includes any previous aspect or combination of aspects, where the fuel comprises hydrogen.

A seventh aspect of the present disclosure includes any previous aspect or combination of aspects, where coke is deposited on the particulate solid that passes from the dehydrogenation reactor to the regeneration unit, and at least a portion of the coke is reacted with oxygen in the regeneration unit.

An eighth aspect of the present disclosure includes any previous aspect or combination of aspects, where passing the particulate solid from the dehydrogenation reactor to the regeneration unit comprises passing the particulate solid through a pre-oxidation unit, where the particulate solid is exposed to an oxygen-containing gas in the pre-oxidation unit such that the content of oxygen in the oxygen-carrier material of the particulate solid is increased in the pre-oxidation unit.

A ninth aspect of the present disclosure includes any previous aspect or combination of aspects, where the particulate solid and the oxygen-containing gas flow generally co-currently in an upward direction through the pre-oxidation unit.

A tenth aspect of the present disclosure includes any previous aspect or combination of aspects, where the regeneration unit further comprises a third gas inlet beneath the first gas inlet and above the second gas inlet and a fourth gas inlet beneath the second gas inlet, where a stripping gas enters the regeneration unit through the third gas inlet and the fourth gas inlet.

An eleventh aspect of the present disclosure includes any previous aspect or combination of aspects, where a portion of the particulate solid is withdrawn from the regeneration unit and passed through at least a portion of the regeneration unit a second time before it is passed to the dehydrogenation reactor.

A twelfth aspect of the present disclosure includes any previous aspect or combination of aspects, where the withdrawn portion of the particulate solid is withdrawn from the regeneration unit from the fuel zone, the first strip zone, the air zone, the second strip zone, or combinations thereof.

A thirteenth aspect of the present disclosure includes any previous aspect or combination of aspects, where the particulate solid consists essentially of the oxygen-carrier material and the dehydrogenation of the one or more hydrocarbons is by non-catalytic thermal dehydrogenation.

A fourteenth aspect of the present disclosure includes any previous aspect or combination of aspects, where the particulate solid further comprises a dehydrogenation catalyst material and the dehydrogenation of the one or more hydrocarbons is at least partially by catalytic dehydrogenation.

A fifteenth aspect of the present disclosure includes any previous aspect or combination of aspects, where the dehydrogenation catalyst material and the oxygen-carrier material are separate particles of the particulate solid or are contained in the same particles of the particulate solid.

It will be apparent to those skilled in the art that various modifications and variations can be made to the presently disclosed technology without departing from the spirit and scope of the technology. Since modifications combinations, sub-combinations and variations of the disclosed embodiments incorporating the spirit and substance of the presently disclosed technology may occur to persons skilled in the art, the technology should be construed to include everything within the scope of the appended claims and their equivalents. Additionally, although some aspects of the present disclosure may be identified herein as preferred or particularly advantageous, it is contemplated that the present disclosure is not limited to these aspects.

It is noted that the various details described in this disclosure should not be taken to imply that these details relate to elements that are essential components of the various embodiments described in this disclosure, even in cases where a particular element is illustrated in each of the drawings that accompany the present description. Unless specifically identified as such, no feature disclosed and described herein should be construed as “essential”. Contemplated embodiments of the present technology include those that include some or all of the features of the appended claims.

For the purposes of describing and defining the present disclosure it is noted that the term “about” are utilized in this disclosure to represent the inherent degree of uncertainty that may be attributed to any quantitative comparison, value, measurement, or other representation. The term “about” are also utilized in this disclosure to represent the degree by which a quantitative representation may vary from a stated reference without resulting in a change in the basic function of the subject matter at issue.

In relevant cases, where a composition is described as “comprising” one or more elements, embodiments of that composition “consisting of” or “consisting essentially of” those one or more elements is contemplated herein.

It should be appreciated that compositional ranges of a chemical constituent in a stream or in a reactor should be appreciated as containing, in some embodiments, a mixture of isomers of that constituent. For example, a compositional range specifying butene may include a mixture of various isomers of butene. It should be appreciated that the examples supply compositional ranges for various streams, and that the total amount of isomers of a particular chemical composition can constitute a range.

It is noted that one or more of the following claims and the detailed description utilize the terms “where” or “wherein” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.”

In some embodiments, chemicals or chemical streams are described as “passing” from one system unit or portion of a system unit to another. As described herein, such passing may include direct passing or indirect passing. For example, when passing from “unit A” to “unit B”, direct passing has no intermediate destination between unit A and unit B (i.e., directly through a pipe or other transport passage), and indirect passing may include one or more intermediate destinations between unit A and unit B. For example, a stream passing from unit A to unit B may passed through, without limitation, a heat exchanger, treatment device, etc.

It should be understood that any two quantitative values assigned to a property may constitute a range of that property, and all combinations of ranges formed from all stated quantitative values of a given property are contemplated in this disclosure. Where multiple ranges for a quantitative value are provided, these ranges may be combined to form a broader range, which is contemplated in the embodiments described herein.

1. A method for dehydrogenating hydrocarbons, the method comprising:
contacting a feed stream comprising one or more hydrocarbons with a particulate solid in a dehydrogenation reactor, wherein the particulate solid comprises an oxygen-carrier material, and wherein in the dehydrogenation reactor:
the one or more hydrocarbons are dehydrogenated to form hydrogen and one or more products; and
at least a portion of the hydrogen is reacted with oxygen from the oxygen-carrier material to form water and reduce the oxygen content in the oxygen-carrier material;
passing the particulate solid from the dehydrogenation reactor to a regeneration unit, wherein:
the particulate solid moves in a generally downward direction through the regeneration unit and gases move in a generally upwards direction through the regeneration unit, such that the particulate solid and gases move in a countercurrent flow pattern through the regeneration unit;
the regeneration unit comprises a first gas inlet and a second gas inlet, the second gas inlet is beneath the first gas inlet, an oxygen-containing gas enters into the regeneration unit through the first gas inlet, and a fuel enters into the regeneration unit through the second gas inlet;
in an area of the regeneration unit above the second gas inlet and beneath the first gas inlet, at least a portion of the fuel is reacted with oxygen from the oxygen-carrier material of the particulate solid; and
passing at least a portion of the particulate solid from the regeneration unit to the dehydrogenation reactor.

2. The method of claim 1, wherein in an area of the regeneration unit above the first gas inlet, the oxygen content in at least a portion of the oxygen-carrier material of the particulate solid increases.

3. The method of claim 1, wherein at least a portion of the fuel passes to the area of the regeneration unit above the first gas inlet, and in the area of the regeneration unit above the first gas inlet at least a portion of the fuel is reacted with oxygen from the oxygen-containing gas.

4. The method of claim 1, wherein the one or more hydrocarbons comprise an alkyl moiety and the one or more products comprise one or more olefinic compounds.

5. The method of claim 1, wherein the one or more hydrocarbons comprise ethane and the one or more products comprise ethylene.

6. The method of claim 1, wherein the fuel comprises hydrogen.

7. The method of claim 1, wherein coke is deposited on the particulate solid that passes from the dehydrogenation reactor to the regeneration unit, and at least a portion of the coke is reacted with oxygen in the regeneration unit.

8. The method of claim 1, wherein passing the particulate solid from the dehydrogenation reactor to the regeneration unit comprises passing the particulate solid through a pre-oxidation unit, wherein the particulate solid is exposed to an oxygen-containing gas in the pre-oxidation unit such that the content of oxygen in the oxygen-carrier material of the particulate solid is increased in the pre-oxidation unit.

9. The method of claim 8, wherein the particulate solid and the oxygen-containing gas flow generally co-currently in an upward direction through the pre-oxidation unit.

10. The method of claim 1, wherein:
the regeneration unit further comprises:
a third gas inlet beneath the first gas inlet and above the second gas inlet; and
a fourth gas inlet beneath the second gas inlet; and
a stripping gas enters into the regeneration unit through the third gas inlet and fourth gas inlet.

11. The method of claim 1, wherein a portion of the particulate solid is withdrawn from the regeneration unit and passed through at least a portion of the regeneration unit a second time before being passed to the dehydrogenation reactor.

12. The method of claim 11, wherein the withdrawn portion of the particulate solid is withdrawn from the regeneration unit from the fuel zone, the first strip zone, the air zone, the second strip zone, or combinations thereof.

13. The method of claim 1, wherein the particulate solid consists essentially of the oxygen-carrier material and the dehydrogenation of the one or more hydrocarbons is by non-catalytic thermal dehydrogenation.

14. The method of claim 1, wherein the particulate solid further comprises a dehydrogenation catalyst material and the dehydrogenation of the one or more hydrocarbons is at least partially by catalytic dehydrogenation.

15. The method of claim 14, wherein the dehydrogenation catalyst material and the oxygen-carrier material are separate particles of the particulate solid or are contained in the same particles of the particulate solid.
